(19) United States
(12) Patent Application Publication
(10) Pub. No.: US 2026/0306015 A1
(43) Pub. Date: Oct. 8, 2026

(54) SYSTEMS AND METHODS FOR DIAGNOSING AND TREATING NASAL CONDITIONS USING TISSUE BIOMARKERS

(71) Applicant: Aerin Medical Inc., Mountain View, CA (US)

(72) Inventors: Scott J. Wolf, Menlo Park, CA (US); Desiree Hollemon, Mountain View, CA (US)

(73) Assignee: Aerin Medical Inc., Mountain View, CA (US)

(21) Appl. No.: 19/639,042

(22) Filed: Apr. 3, 2026

Related U.S. Application Data
(60) Provisional application No. 63/783,198, filed on Apr. 3, 2025.

(51) Int. Cl. A61B 5/00; A61B 5/145; A61B 17/00; A61B 18/00; A61B 18/12; A61B 18/14; G16H 20/30; G16H 50/30

(52) U.S. Cl. CPC A61B 5/4836; A61B 5/14503; A61B 5/14546; A61B 18/1485; G16H 20/30; G16H 50/30; A61B 2017/00154; A61B 2018/0016; A61B 2018/00327; A61B 2018/00577; A61B 2018/00613; A61B 2018/1266

(57) ABSTRACT

Systems and methods are provided for treating tissue in a nasal airway. In some embodiments, a method for treating tissue is provided that includes assessing one or more biomarker profiles in nasal tissue, the one or more biomarker profiles being indicative of rhinitis, advancing a treatment device into a nasal cavity of a patient, the treatment device including one or more treatment elements, the one or more treatment elements having an array of energy delivery elements disposed thereon, and providing treatment, based on the assessed biomarker profiles, to one or more target tissue areas in the nasal cavity by activating one or more of the energy delivery elements of the array of energy delivery elements. The one or more biomarker profiles relate to tyrosine hydroxylase (TH), choline acetyltransferase (ChAT), neuropeptide Y (NPY), calcitonin gene-related peptide (CGRP), neurokinin A (NKA), neurokinin B (NKB), or substance-P (SP).

### RELATED APPLICATIONS

This application claims the benefit of and priority to U.S. Provisional Application No. 63/783,198, filed Apr. 3, 2025, the contents of which is hereby incorporated herein by reference in its entirety

### FIELD

The present application is related to medical devices and methods, and more specifically, relates to a system and method for treating nasal airway tissue using tissue biomarkers to diagnose rhinitis.

### BACKGROUND

Therefore, it would be desirable to have improved devices, systems and methods for treating chronic and allergic rhinitis. Ideally, such devices, systems and methods would have longer term effects than medications and be less invasive than currently available surgical techniques.

### SUMMARY

In some embodiments, a method for treating tissue is provided that includes assessing one or more biomarker profiles in nasal tissue, the one or more biomarker profiles being indicative of rhinitis; advancing a treatment device into a nasal cavity of a patient, the treatment device including one or more treatment elements, the one or more treatment elements having an array of energy delivery elements disposed thereon; and providing treatment, based on the assessed biomarker profiles, to one or more target tissue areas in the nasal cavity by activating one or more of the energy delivery elements of the array of energy delivery elements; wherein the one or more biomarker profiles relate to tyrosine hydroxylase (TH), choline acetyltransferase (ChAT), neuropeptide Y (NPY), calcitonin gene-related peptide (CGRP), neurokinin A (NKA), neurokinin B (NKB), or substance-P (SP).

In some embodiments, the method further includes monitoring the one or more biomarker profiles during the treatment to determine a need for additional treatment with the treatment device.

In some embodiments, the method further includes monitoring the one or more biomarker profiles after termination of the treatment to determine a need for additional treatment with the treatment device.

In some embodiments, the method further includes monitoring the one or more biomarker profiles after termination of the treatment to access results of the treatment with the treatment device.

In some embodiments, one or more energy delivery elements of the array of energy delivery elements are activated with pulsed electric field energy for conducing pulsed radiofrequency energy or pulsed field ablation. In some embodiments, one or more energy delivery elements of the array of energy delivery elements are activated with high-voltage direct current pulses of short duration to induce regulated cell death through irreversible electroporation.

In some embodiments, the array of energy delivery elements is positioned in two or more rows on the one or more treatment elements. In some embodiments, each of the two or more rows include at least one pair of energy delivery elements.

In some embodiments, a method for treating tissue is provided that includes assessing one or more biomarker profiles in nasal tissue, the one or more biomarker profiles being indicative of rhinitis; providing an initial treatment, based on the assessed biomarker profiles, to one or more target tissue areas in a nasal cavity by activating one or more energy delivery elements positioned on a treatment element of a treatment device; monitoring the one or more biomarker profiles during the initial treatment to track the initial treatment provided with the one or more energy delivery elements of the treatment device; and providing additional treatment, based on the monitored biomarker profiles during the initial treatment, to the one or more target tissue areas in a nasal cavity, wherein the one or more biomarker profiles relate to tyrosine hydroxylase (TH), choline acetyltransferase (ChAT), neuropeptide Y (NPY), calcitonin gene-related peptide (CGRP), neurokinin A (NKA), neurokinin B (NKB), or substance-P (SP).

In some embodiments, the one or more energy delivery elements are activated with pulsed electric field energy for conducing pulsed radiofrequency energy or pulsed field ablation.

In some embodiments, the one or more energy delivery elements are activated with high-voltage direct current pulses of short duration to induce regulated cell death through irreversible electroporation.

In some embodiments, the one or more energy delivery elements are positioned in two or more rows on the treatment element. In some embodiments, each of the two or more rows includes at least one pair of energy delivery elements.

In some embodiments, the method further comprising accessing the one or more biomarker profiles after providing the additional treatment.

In some embodiments, a method for treating tissue is provided that includes assessing a first set of biomarker profiles in nasal tissue, the first set of biomarker profiles being indicative of rhinitis; providing treatment by applying energy to one or more target tissue areas in a nasal cavity with one or more pairs of electrodes; assessing a second set of biomarker profiles in nasal tissue after providing the treatment to the one or more target tissue areas; and comparing the first set of biomarker profiles to the second set of biomarker profiles to monitor the treatment to determine success of the treatment in treating rhinitis, wherein the biomarker profiles relate to tyrosine hydroxylase (TH), choline acetyltransferase (ChAT), neuropeptide Y (NPY), calcitonin gene-related peptide (CGRP), neurokinin A (NKA), neurokinin B (NKB), or substance-P (SP).

In some embodiments, the one or more pairs of electrodes are activated with pulsed electric field energy for conducing pulsed radiofrequency energy or pulsed field ablation.

In some embodiments, the one or more pairs of electrodes are activated with high-voltage direct current pulses of short duration to induce regulated cell death through irreversible electroporation.

In some embodiments, the second set of biomarker profiles being higher than the first set of biomarker profiles indicates success of the treatment.

In some embodiments, the method further includes providing additional treatment to the one or more target tissue areas in a nasal cavity when the comparison of the first set of biomarker profiles to the second set of biomarker profiles determines the treatment is unsuccessful.

In some embodiments, the method further includes determining success of the treatment when the comparison of the first set of biomarker profiles to the second set of biomarker profiles indicates that the biomarker profiles have increased to above a threshold value or decreased to below a threshold.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 illustrates an exemplary flowchart of a method for diagnosing a condition of a nasal cavity;

FIG. 2 illustrates an exemplary flowchart of a method for treating a condition of the nasal cavity;

FIG. 3A illustrates an exemplary embodiment of a system for treating nasal airway tissue;

FIG. 3B is a front perspective view of an electrosurgical system for treating nasal airway tissue, according to one embodiment;

FIGS. 4A and 4B illustrate exemplary embodiments of a system for treating tissue having a plurality of output channel for delivering energy to tissue;

FIGS. 5A and 5B are exemplary embodiments of side and top perspective views, respectively, of a distal tip (or “treatment element”) of a treatment device or stylus;

FIGS. 6A and 6B are perspective views of a distal end of a stylus, illustrating operation of the expandable wire electrode component;

FIG. 7 illustrates an embodiment of a nasal airway tissue treatment stylus that includes an expandable wire electrode component that advances out of the distal tip of the device;

FIGS. 8A and 8B illustrate exemplary embodiments of a treatment device having an expandable wire electrode component;

FIG. 9 illustrates an exemplary processing architecture;

FIG. 10A illustrates an exemplary flow diagram of an example method of use of the various devices disclosed herein;

FIG. 10B illustrates an exemplary embodiment of advancing a portion of the device to a treatment site in the nasal cavity;

FIG. 10C is a sagittal view of a human nasal cavity showing an exemplary embodiment of multiple treatments performed on nasal tissue with a multichannel device;

FIGS. 11A and 11B illustrate the mechanism of electroporation and cell death that can be achieved for treating rhinitis;

FIGS. 12A and 12B show an exemplary cross-sectional view of the nasal cavity;

FIGS. 13A, 13B, 13C, and 13D illustrate anatomical elements of a human nose; and

FIG. 13E is a sagittal view of a human nasal cavity, illustrating the lateral wall and nasal nerves

FIGS. 14A, 14B, 14C, and 14D show computed tomography (CT) and endoscopic images to illustrate the nasal nerves;

FIG. 15 shows representative intraneural staining with CGRP in PLNNs across different cadavers;

FIG. 16 shows representative intraneural NKA staining being equivalent across different nasal nerve types in different cadavers;

FIGS. 17A, 17B, 17C, and 17D show examples of immunostaining for different neuropeptides in posterolateral nasal nerves (PLNN) across different cadavers;

FIGS. 18A, 18B, 18C, and 18D show a comparison of neuropeptide staining relative to the appearance of the same nerves on hematoxylin and eosin staining;

FIGS. 19A, 19B, 19C, and 19D show a comparison of neuropeptide staining relative to the appearance of the same nerves on hematoxylin and eosin staining; and

FIG. 20 shows Supplemental Table I.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following disclosure provides embodiments of systems and methods for diagnosing and assessing treatment of conditions (such as nasal conditions), including, for example, rhinitis, craniofacial pain, and migraines, improving breathing, airway inflammation, chronic rhinosinusitis with and without nasal polyps, and sleep disorders. The diagnosis and treatment of nasal conditions can employ the measurement and tracking of tissue biomarkers.

During use of any of the embodiments of a treatment device described herein, the system can be used to assess and track biomarkers in the nasal tissue that are indicative of rhinitis or other diseases associated with the nasal tissue. The biomarker information can used to determine the need for treatment, treatment success, or need for additional treatment. The analysis can occur in before, during, or after delivery of energy for nasal tissue treatment with a treatment device. It should be noted that while the systems and methods of the present disclosure are described in connection with nasal tissue, the systems and methods of the present disclosure may be used in connection with other types of tissue.

In some embodiments, the information related to biomarkers can be displayed to the user before, during, or after treatment with the treatment device. The biomarker measurements can be analyzed, for example, using software, to obtain information to help the user to determine whether intervention or treatment is necessary or desirable. In some embodiments, the biomarker measurements may be communicated directly to a computer system.

In some embodiments, the biomarker data, such as data relating to neurokinin-A (NKA), can be acquired and analyzed to provide information about the tissue before, during or after tissue treatment including, but not limited to, progression of the treatment for rhinitis or other diseases. For example, when treatment includes a plurality of application of energy to the tissue, biomarker information can be obtained between one or more applications of the energy to monitor the treatment as the biomarker information changes as treatment progress. In some embodiments, data regarding biomarkers can be analyzed and presented visually to the user via a graphical user interface in a way that provides the user with feedback regarding the changes in the biomarkers or treatment progression using the biomarker assessment information. In some embodiments, the biomarkers are at least NKA. In some embodiments, the biomarkers are at least tyrosine hydroxylase (TH), choline acetyltransferase (ChAT), neuropeptide Y (NPY), calcitonin gene-related peptide (CGRP), neurokinin B (NKB), or substance-P (SP).

In some embodiments, a change in the biomarker profiles of one or more biomarkers can be indicative of the success of the treatment delivered to the tissue. For example, the biomarker data can increase or decrease. In some embodiments, the need for additional treatment can be determined based on the change in the biomarker profile based on a comparison of the biomarker profiles accessed at different times before, during, or after treatment. For example, a first set of biomarker profiles can be measured before treatment (for example, before a first or initial treatment) and can be compared to a second set of biomarker profiles taken during or after treatment to determine if the treatment is successful. A threshold value of change can be used to determine if the treatment was successful. A second or additional treatment can be delivered to the tissue if needed.

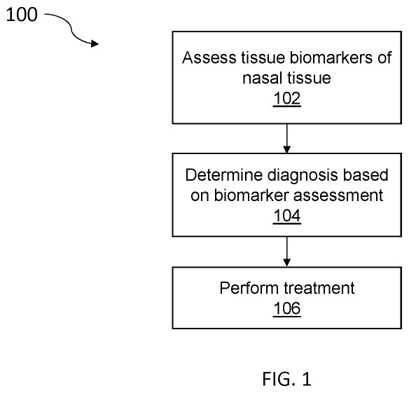

FIG. 1 is an exemplary flow diagram of an example method of diagnosing nasal conditions based on biomarker data. In some embodiments, biomarker information is used to diagnose a condition of the nasal cavity, such as rhinitis. In some embodiments, the method 100 can include assessing one or more tissue biomarkers of nasal tissue in step 102. In step 104, a diagnosis is determined based on biomarker assessment. In step 106, treatment can be performed based on the diagnosis.

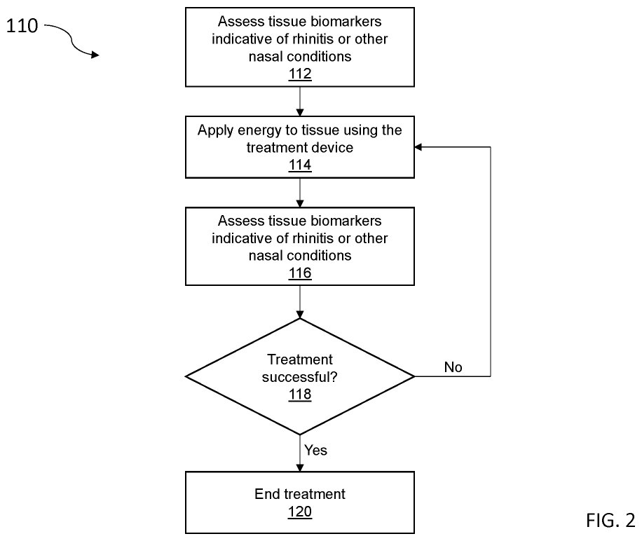

FIG. 2 is an exemplary flow diagram of an example method of use of the various devices disclosed herein to treat nasal conditions while tracking biomarker indications in the tissue. In some embodiments, the biomarker information can be used to track treatment or determine treatment success. In some embodiments, the method 110 may include assessing one or more tissue biomarkers in step 112, and delivering therapy, for example by applying energy, to the treatment site in step 114.

After therapy is delivered, the biomarkers can be assessed (in step 116) to make a determination on the success of the treatment (in step 118). For example, the biomarker profiles or data accessed prior to the treatment can be compared to the biomarker profiles or data collected during or after the treatment to monitor the treatment to determine success of the treatment, for example, in treating rhinitis If, based on the assessment in step 108, that the treatment was successful, the treatment device may be removed from the nasal cavity in step 120. Additional therapy can be delivered if the assessment of the biomarkers indicates that further treatment is necessary.

This application describes various aspects and embodiments of a device, system, and method for treating nasal airway tissue. For example, the devices described herein can be used for nerve destruction for posterior nasal nerve treatment for treatment of chronic rhinitis, treatment of migraines through nasal nerve treatments, or other diseases that can be treated with nerve ablation that are in the ears, nose, or throat.

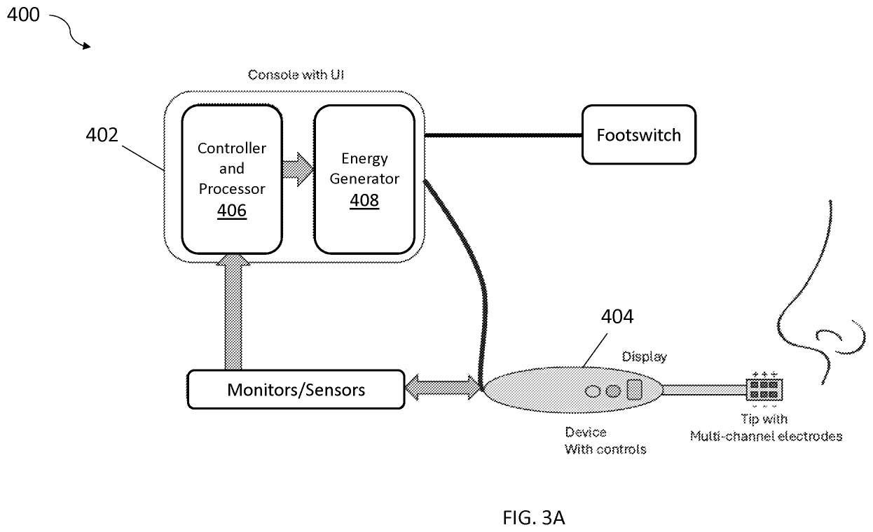

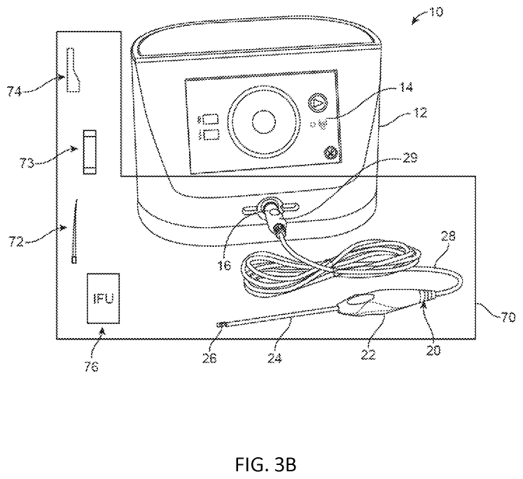

In some embodiments, the system can include a console with an electrosurgical generator and a display, and a treatment device, or stylus, coupled to the console via a cable, as shown in FIGS. 3A and 3B. In some embodiments, the electrosurgical generator is configured to provide radiofrequency (RF) energy and can be delivered continuously or through short duration pulses to minimize the thermal effect on adjacent tissue. In some embodiments, the electrosurgical generator is configured to provide pulsed electric field (PEF) energy for conducing pulsed radiofrequency energy (PRF) or pulsed field ablation (or “PEF ablation”) of one or more tissues. In some embodiments, the electrosurgical generator is configured to provide high-voltage direct current pulses of short duration to induce regulated cell death (e.g. apoptosis) through irreversible electroporation, this treatment modality has minimal thermal effect and can target specific tissues while minimizing collateral damage. In some embodiments, the system may be configured to deliver another type of energy, such as but not limited to heat, laser, microwave, cryogenic cooling, DC current, or ultrasound.

Referring again to FIG. 3A, FIG. 3A illustrates an exemplary embodiment of a nasal airway treatment system 400 that includes a console 402 for controlling energy delivery to a plurality of pairs of electrodes on a treatment device 404 or stylus and displaying information to the user. The console includes a controller 406 and an energy generator 408. In some embodiments, the energy generator is a pulsed field generator for producing pulsed energy, such as PFA or PRF.

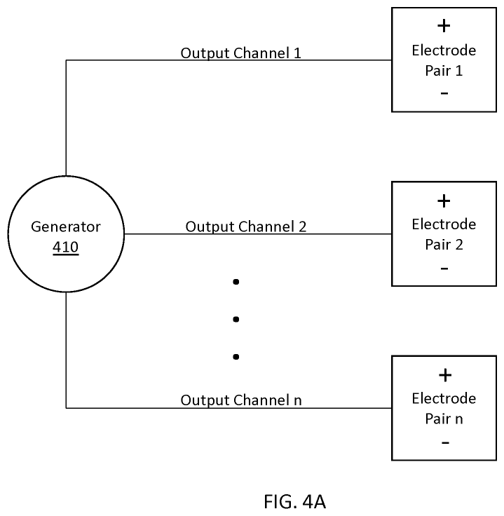

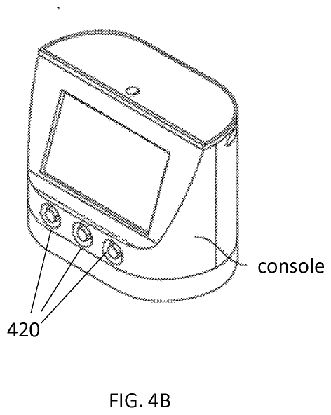

In some embodiments, the console includes one or more ports, or output channels, for connecting to the treatment device or stylus, for treating one or more target tissues in the nasal cavity with energy. In some embodiments, each pair of electrodes on the treatment device can be connected to a separate, controlled electrical output channel to allow for different electrode pairs of the treatment element to be activated separately and independently of one another, as shown in FIGS. 4A and 4B. For example, FIG. 4A illustrates a generator 410 that comprises output channel 1 through output channel n for delivering energy to electrodes. For example, FIG. 4B illustrates a console that includes a plurality of output channels 420 for delivering energy. By controlling the treatment energy flowing through each pair of electrodes using parameters including, but not limited to, temperature, a greater degree of control and accuracy over the treatment energy may be obtained, such that treatments may be repeatable to each electrode pairs or subsets of electrode pairs. In some embodiments, each of the plurality of output channels can be assigned to an individual electrode pair. In some embodiments, each of the plurality of output channels can be assigned to a subset of the plurality of electrode pairs.

More specifically, in some embodiments of treatment devices comprising an array or multiple pairs of electrodes, each pair of electrodes (bipolar) or each electrode (monopolar) may have a separate, controlled electrical output channel to allow for different regions of the treatment element to be activated separately.

The output channels may also comprise separate or integrated feedback. This may allow for more accurate temperature control and more precise targeting of tissue. Separate control may also allow energy to be focused or intensified on a desired region of the treatment element in cases where the anatomy of the nasal tissue/structures does not allow the entire electrode region of the treatment element to engage the tissue. In such embodiments, the nasal tissue that is in contact with the treatment element may receive sufficient energy to treat the tissue.

Referring again to FIG. 3B, in some embodiments, an exemplary embodiment of a nasal airway tissue treatment system 10 includes two primary components: a console 12 and a treatment device or stylus 20. Similar to the console shown above, the console 12 includes a display 14, an electrosurgical generator, and electronics (inside the console 12 and thus not visible), and an outlet 16 into which the stylus 20 is plugged. The stylus 20 includes a handle 22, a shaft 24, a distal tip 26 (also referred to as a “treatment element”), a cable 28 and a connector 29 for connecting the stylus 20 with the outlet 16. Many features of the console 12 and the system 10 in general are described further below, in U.S. patent application Ser. No. 16/668,678, and in the other Incorporated References. In some embodiments, the electrosurgical generator is configured to provide pulsed electric field (PEF) energy for conducing pulsed field ablation (or “PEF ablation”) of one or more tissues, including but not limited to any suitable nerve tissue.

The console 12 is a reusable device, which is designed and intended for use with multiple patients. The stylus 20, on the other hand, is a single-patient, single-use, disposable device. In some embodiments, the stylus 20 may be provided as part of a stylus kit 70, which may include a curved anesthesia needle 72, a packet of conductive gel 73, a shaft bending tool 74 or instructions for use 76 (or “IFU”). Anesthesia protocols for anesthetizing the patient's nasal cavity are largely up to the physician, and many different protocols are known to otolaryngologists. In some embodiments, it may be required or strongly recommended to inject anesthetic into the mucosa or submucosa in the target area(s), in order to help direct the current delivered by the stylus 20. This may be helpful in some embodiments, because fluid such as anesthetic is generally conductive for RF. All of these kit components are optional, and any embodiment of the stylus kit 70 may include fewer items or additional items, without departing from the scope of the invention. In some embodiments, the stylus 20 may be provided by itself for use with the console 12. The stylus 20 may be used for multiple treatments on the same patient at the same time—for example multiple treatment areas in a nostril or treatment of both nostrils—and then is disposed of after use on that patient. In some embodiments, the stylus 20 may be sterilizable and reusable.

The stylus shaft 24 may have any length suitable for reaching the posterior portion of the nasal cavity where the posterior nasal nerves reside. In some embodiments, for example, the shaft 24 is approximately 3.75 inches long, from its connection to the distal tip 26 at one end to its connection with the handle 22 at the opposite end. In some embodiments, the shaft 24 may have different lengths, for example between about 3 inches and about 4 inches in embodiments designed for addressing a posterior portion of the nasal cavity, and between about 1 inch and about 3 inches in embodiments designed for addressing an anterior portion of the nasal cavity, such as the nasal valve. The shaft 24 may also have any suitable width (or “diameter”). For example, in some embodiments, the shaft may have a width of about 4 mm to about 5 mm. The shaft 24 will be described in further detail below.

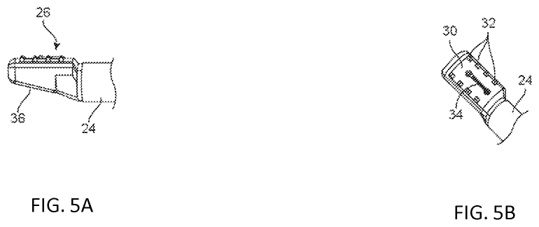

Referring now to FIGS. 5A and 5B, the distal tip 26 is shown in detail. The distal tip 26 includes a treatment surface 30 (or “tip face”), two rows of four bipolar RF electrodes 32, and a thermocouple 34 between the electrodes 32. As illustrated in FIG. 5A, the bottom surface 36 of the distal tip 26 may be tapered or slanted, to give the distal end of the distal tip 26 a narrower profile to facilitate insertion and advancement of the distal tip 26 into the nostril and to the posterior portion of the nasal cavity. The treatment surface 30 is made of a non-conductive material (e.g., plastic) and may have a slightly convex shape, as illustrated, or may alternatively be flat, concave, or more convex than the shown in FIG. 5B. The slightly convex shape may help ensure contact of the electrodes 32 and the thermocouple 34 with mucosal tissue during use.

In some embodiments, the thermocouple 34 may be removed or replaced by a different type (or shape or number) of temperature sensor(s). The system can use one or more temperature sensors to monitor temperature of the treated tissue or adjacent tissues to monitor for changes or to execute a tissue apposition check or monitor other biometric information. The thermocouple 34 (or any temperature sensor) is used to measure temperature of the mucosa and provide the measurements to a processor in the console 12, which can in turn automatically adjust whether and how much energy is delivered to the stylus 20. In some embodiments, the distal tip 26 or a portion thereof may be translucent, to facilitate visualization of target tissue or nasal mucosa.

The system can also monitor the impedance of the tissues in contact with the device to monitor for changes or to execute a tissue apposition check or monitor other biometric information. The system may include an optical generator that enables an optical signal, possibly in the near infrared spectrum, to be sent to the tissue and enables monitoring of the treated tissues by confirming tissue has been altered through some property shift, for example, a decrease in oxygenation may indicate that irreversible electroporation has occurred in the targeted tissue.

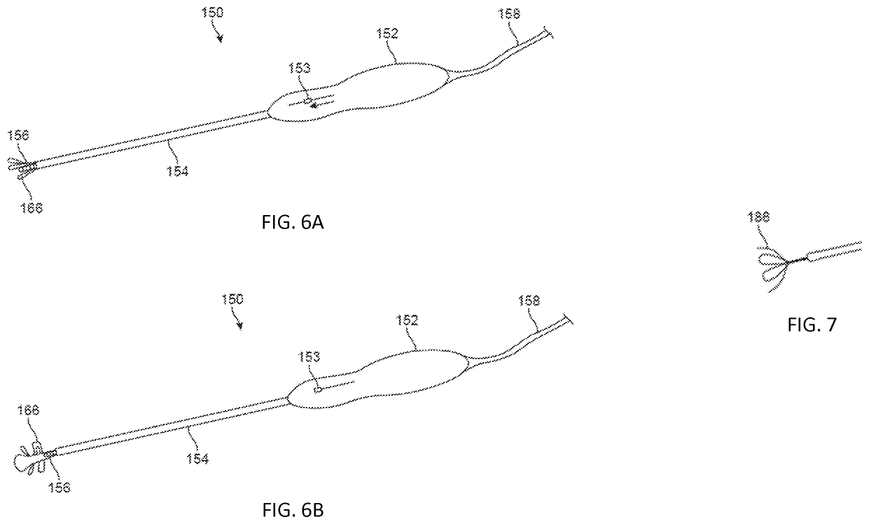

Referring now to FIGS. 6A and 6B, another embodiment of a nasal airway tissue treatment element is illustrated. The treatment element can be in the form of an expandable wire electrode component 166 that can be moved out of the distal end of the shaft 154. FIG. 6B shows the slider 153 further advanced and the expandable wire electrode component 166 fully advanced and expanded. The expandable wire electrode component 166 may include multiple bipolar electrode pairs located at various locations along its length. Optionally, the expandable wire electrode component 166 may also include one or more thermocouples or multiple nerve sensors (such as electrodes), which may be used to measure a temperature of nasal mucosa and sense where nerve tissue is located under the mucosa, respectively. In some cases, all electrode pairs may be activated at the same time. Alternatively, only one or more selected electrode pairs may be activated. In some cases, the expandable wire electrode component 166 may be used to determine the location of nerves and then only bipolar electrode pairs located directly over those nerves may be activated.

In that configuration, the expandable wire electrode component 166 may be used to treat posterior nasal nerves or any other nerve tissue the physician wants to treat. This is only one example, however, and a physician may use the stylus 150 in any suitable manner. After treatment with the expandable wire electrode component 166 is complete, the expandable wire electrode component 166 may be pulled back into the shaft by sliding the slider 153 proximally.

With reference now to FIG. 7, another alternative embodiment of a treatment element in the form of an expandable wire electrode component 186. In some embodiments, the expandable wire electrode component 186 is located inside an opening and the lumen (not visible on these drawings) and may be advanced out of the opening to allow the expandable wire electrode component 186 to expand and be used for treatment. All of the same features and methods of use described above may be applied to this embodiment of the stylus, the primary difference being that the expandable wire electrode component 186 is located within the distal tip and advances out of the opening. The expandable wire electrode component 186 may have any suitable size, shape and configuration of wires, according to various embodiments.

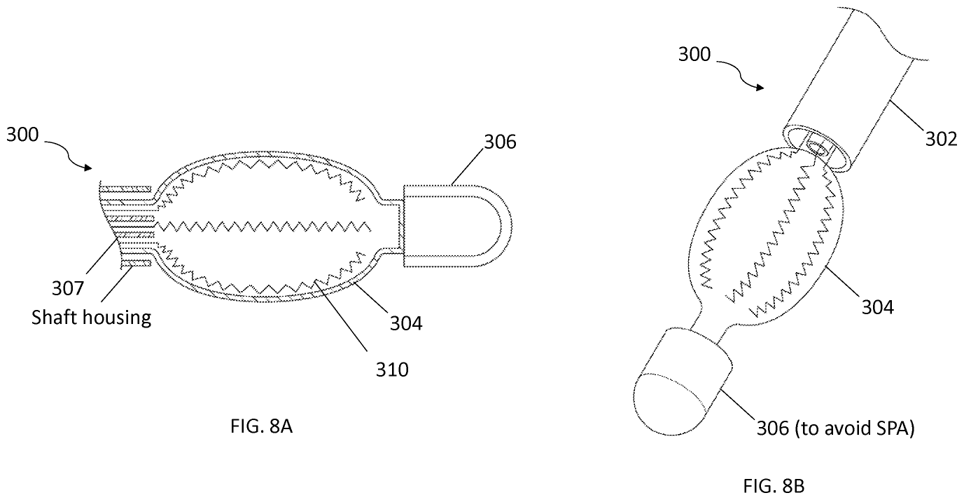

FIGS. 8A and 8B illustrate exemplary embodiments of a treatment device having an expandable wire electrode component with a plurality of electrodes thereon for delivering energy to the target nasal tissue. In some embodiments, the treatment device 300 in is in the form of a sheath balloon. The treatment device 300 includes an elongate sheath with an opening at a distal end such that an expandable member 304, such as a balloon, can extend therefrom. In some embodiments, the balloon is configured to expand asymmetrically. A standoff 306 is couped to a distal end of the balloon, which can in the form of a soft-tipped component or in the form of a wire loop. An inflation lumen 307 extends thought the shaft and couples to a proximal end of the balloon is allow the balloon to move from an uninflated configuration to an inflated configuration (FIGS. 8A and 8B) to allow one or more electrodes 310 positioned on the outer surface of the ballon to contact a target tissue for treatment. The one or more electrodes 310 can be positive or negative and can be symmetrical or asymmetrical. The shape of the electrodes can vary, but the electrodes are configured to expand as the balloon is inflated.

### Methods of Treatment

This application describes various aspects and embodiments of a device, system, and method for treating tissue, including nerve tissue.

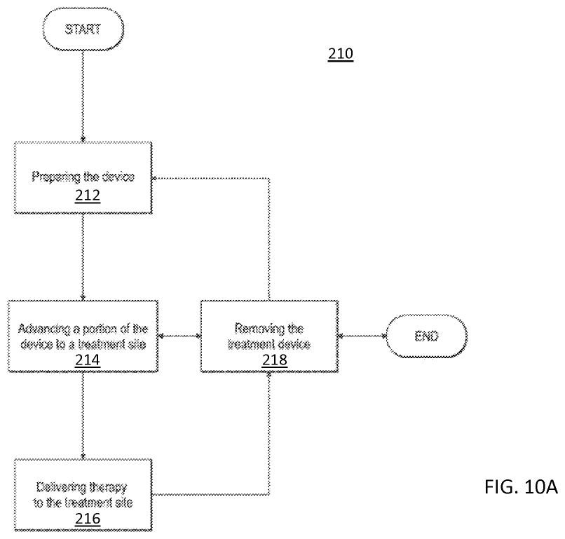

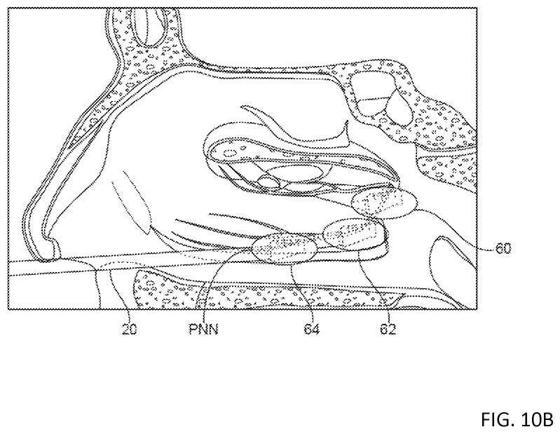

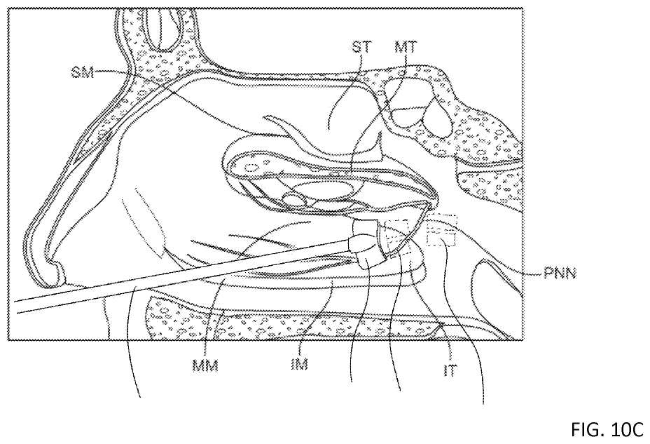

FIG. 10A is a flow diagram of an example method 210 of use of the various devices disclosed herein. In certain implementations, the method 210 may include preparing the device in step 212, advancing a portion of the device to a treatment site in the nasal cavity in step 214, delivering therapy to the treatment site in step 216, and removing the treatment device in step 218.

In some embodiments, step 212 of preparing the device may include removing the device from sterile packaging, assembling one or more components of the device, sterilizing the device, attaching the device to an energy source, or other preparatory work. In certain implementations, this step may include customizing the device to suit the particular needs of the patient and the clinician. This may include articulating, manipulating, or otherwise changing one or more components of the device.

As shown in FIG. 10B, step 214 of advancing a portion of the device to a treatment site in the nasal cavity may follow the preparation of the device in step 212. For example, the clinician may navigate the patient's nasal anatomy to advance a treatment portion of the treatment device into a nostril of the patient to contact mucosal tissue of the upper airway with a treatment surface of the treatment device. Step 214 may be performed without piercing the mucosal tissue. The goal of the navigation may be to place the treatment element in contact with the treatment site.

FIG. 10C shows an exemplary embodiment of multiple treatments applied to target tissue with a multichannel device. Pairs of dashed rectangles show previous treatments.

During navigation, the clinician may perform one or more tests to determine whether proper contact with the treatment site has been made. In some embodiments, the clinician may activate one or more pairs of electrodes. Based on measured results, the clinician may determine that proper contact has not been achieved because an energy pathway could not be made between one or more pairs of electrodes or that one or more measured electrical parameters (e.g., impedance, voltage, current, temperature, etc.) is outside of a desired range. In some embodiments, the clinician may attempt to apply pressure to the treatment site with the treatment portion and determine by feel whether proper contact has been made. In some embodiments, clinician may take a reading using a thermocouple or an electrode to determine whether proper contact has been made. Based on the one or more tests, the clinician may determine that proper contact has been made between the treatment portion and the treatment site. During navigation, a navigation system may be used to position the device based on previously obtained images.

Energy can then be delivered to the treatment site in step 216. As is shown in FIG. 10B, the physician may then move the treatment portion to another treatment site and repeat the steps of confirming contact and delivering energy. For example, as shown in FIG. 10B, the treatment sites include a first target location 60, a second target location 62 and a third target location 64, all of which fall along the path of a posterior nasal nerve PNN. Additional treatments may optionally be provided in the same general area or in different areas of the nasal cavity. In certain circumstances, the clinician may determine that proper contact has not been made and the physician can then either re-orient the treatment portion or move it to another treatment site.

Once the treatment is complete, the treatment device may be removed from the nasal cavity in step 218.

The steps above and much of the disclosure is described with respect to the nose and nasal cavity, but it will be understood that the systems, devices, and methods described herein can be used in other areas of the body, including but not limited to the mouth, including the soft palate and the base of the tongue, the throat, including nerves in the throat, and the ear canal.

In some embodiments, energy used by the systems and devices described herein may be applied in the form of heat, radiofrequency (RF), pulsed radiofrequency, laser, light, ultrasound (e.g., high intensity focused ultrasound), microwave energy, electromechanical, mechanical force, cooling, alternating or direct electrical current (DC current), chemical, electrochemical, cryotherapy, cryogenic, pulsed field ablation, cryoablation spray, lithotripsy, histotripsy, shock wave, or others. In some embodiments, the nasal valve or surrounding tissues may be strengthened through the application of cryogenic therapy, or through the injection or application of bulking agents, glues, polymers, collagen or other allogenic or autogenic tissues, or growth agents.

The systems and methods of the present disclosure can be used to treat various conditions inside or outside of the nasal cavity. Some of the conditions that may be treated by the embodiments described herein include, but are not limited to, rhinitis, asthma, COPD, emphysema, airway inflammation, nasal obstruction or congestion, eye inflammation and allergic conjunctivitis, chronic cough, post nasal drip, allergies, nasal polyps, sinusitis, turbinate enlargement, migraine headaches, other headaches (e.g., cluster headaches, nasal contact point headaches, etc.), tinnitus, dizziness, vertigo, dry eye, excessive tearing, empty nose syndrome, pain (e.g., facial nerve pain, trigeminal neuralgia, complex regional pain syndrome, etc.), sleep apnea, snoring, disordered breathing during sleep, anxiety, mood disorders, middle ear conditions (e.g., otitis media), Eustachian tube dysfunction, herpes zoster, paroxysmal hemicranias, cancer of the head or neck, and reduction of chemical mediators that lead to any of the above-listed conditions. It should be noted that many of these conditions may be co-morbid with one another, and so a treatment of one condition may alleviate one or more other conditions.

By way of non-limiting example, in some embodiments, the systems and methods of the present disclosure can be used to treat or alleviate rhinitis, which can be generally described as the inflammation of the nasal mucosa due to allergic or non-allergic triggers, which leads to excessive mucus production in the nasal cavity. This increased mucus can cause runny nose (rhinorrhea), post nasal drip syndrome (PNDS), upper airway cough syndrome (UACS), congestion, itching, and sneezing symptoms.

In some embodiments, the systems and methods of the present disclosure can be used to decrease mucus production in the upper airways. Mucus hypersecretion often occurs when a mucus producing cell displays rapid mucus secretion behavior. In some embodiments, to prevent this, properties of the cell can be modified to inactivate or retard the cell so as to halt or impede the release of mucins into the airway. In some embodiments, modifying the properties of the mucus producing cells may include reducing the rate at which cells produce mucus. In some embodiments, modifying the properties of mucus producing cells may include deactivating some or all mucus producing cells in a region. In some embodiments, the cells can be modified by being locally destroyed and can be replaced with healthier mucosal tissue, which may mean fewer mucous glands, fewer mucus producing cells, less inflammation, or more normal nerve function. While some embodiments may change a property of a tissue, such as an ability of a tissue to produce mucus, other embodiments may change a tissue from one type to another type. One example of this latter change is changing mucosal tissue containing goblet cells to mucosa with fewer goblet cells or scar tissue. These various embodiments may be used to reduce movement of mucus, reduce the amount of mucus produced, reduce frequency of mucus production, change the mucus viscosity/consistency, or change the path of mucus flow. In some embodiments, the systems and methods of the present disclosure can be used to apply energy to stimulate or disrupt various nasal nerves (as described below) to decrease mucus production in the upper airways.

By way of another non-limiting example, in some embodiments, the systems and methods of the present disclosure can be used to treat or alleviate migraine or headaches, such as cluster headaches, nasal contact point headaches, tension headaches, sinus headaches, or similar conditions. In some embodiments, the systems and methods of the present disclosure can be used to apply energy to stimulate or disrupt various nasal nerves (as described below) to alleviate migraines or headaches.

By way of another non-limiting example, in some embodiments, the systems and methods of the present disclosure can be used to treat or alleviate sleep apnea, disordered breathing during sleep other sleep disorders. In some embodiments, tissues of the soft palate in the mouth can be reshaped, strengthened, stiffened or otherwise altered, including but not limited to skin, muscle, mucosa, submucosa or cartilage of the soft palate. This change in the soft palate can prevent collapse of the soft palate or vibration of the soft palate during nighttime breathing and thus prevent or at least reduce sleep apnea or snoring. One example of this is reducing nasal congestion through treatment of the nasal nerves leading to better breathing during sleep. This also reduces the negative pressure generated in the nasal cavity on inspiration leading to less airway collapse.

By way of another non-limiting example, in some embodiments, the systems and methods of the present disclosure can be used to treat or alleviate nasal airway obstruction, or to increase airflow, by, for example, modifying or reshaping various structures of the nasal cavity, such as the nasal valve area, turbinates, nasal wall, or similar tissue in the nasal cavity. For example, this can be used for improving breathing by decreasing airflow resistance or perceived airflow resistance at or near a site of an internal or external nasal valve. While, in some instances, nasal dysfunction can lead to poor airflow, nasal breathing can also be improved in people with normal breathing or normal nasal anatomy by decreasing nasal airflow resistance in the nasal valve and associated nasal anatomy. Remodeling or changing the structure of the nasal valve can improve nasal airflow in people with inadequate nasal airflow resulting from causes other than nasal valve dysfunction, such as a deviated septum, enlarged turbinates, mucosal swelling, or mucus overproduction.

By way of another non-limiting example, in some embodiments, the systems and methods of the present disclosure can be used for mucosal resurfacing or remodeling by delivering stimulus to mucosal tissue. A stimulus can be applied to cause local destruction of the mucosal layer and replacement with healthier mucosal tissue, which may mean fewer mucous glands, fewer mucus producing cells, less inflammation, fewer inflammatory cells, or more normal nerve function.

In some embodiments, the systems and methods of the present disclosure can be used to effect mucosal remodeling through direct stimulus from an agent or a stimulus. The resurfacing of the mucosa replaces the diseased or chronically inflamed mucosal cells with healthy mucosa that has more normal function, such as producing less mucus or other fluids, being less prone to allergy, and having less nerve stimulation or less activity of inflammatory cells. In some embodiments, this can be through modification of the cell types that comprise the mucosal layers. In some embodiments, this can include removing nerve ending present in the mucosal layer.

In some embodiments, the systems and methods of the present disclosure can be used to apply energy to submucosal tissue, such as mucus cells and glands. In some embodiments, the energy is applied to the posterior region of the nasal cavity.

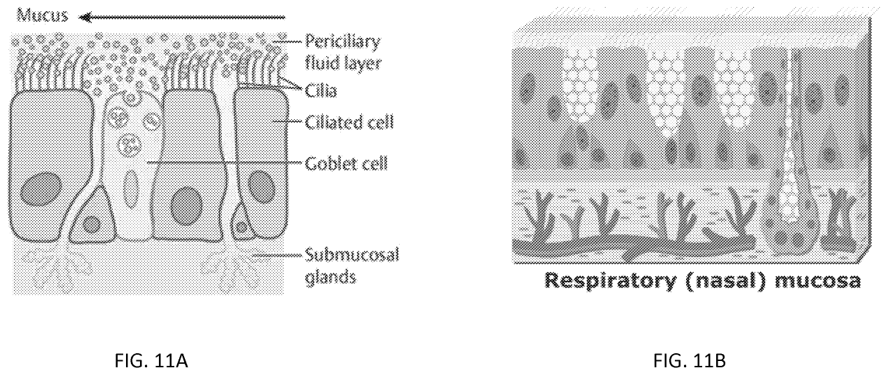

FIG. 11A and FIG. 11B show an exemplary cross-sectional view of the mucosa, according to one or more embodiments herein. In reference to FIGS. 11A and 11B, the stimulus can be delivered at a desired depth deeper than the thickness of the mucosa. The mucosal layer of the nasal cavity is between 0.5 mm and 5.0 mm thick, with an average thickness of 3.5 mm. In some embodiments, the energy is applied within submucosal tissue to destroy or vary a function of ciliated cells, goblet cells, or other epithelial cells of the nasal cavity, or mucous or serous glands.

In some embodiments, the systems and methods of the present disclosure can be used to apply a stimulus to neuromodulate certain nerves within the nasal cavity. In some embodiments, any nerve or combination of nerves in the nasal cavity can be treated to treat various conditions inside or outside of the nasal cavity. Generally, nasal neuromodulation may involve treating one or more nerves to change or disrupt their functions without completely stopping their function. At the same time, however, nasal neuromodulation may involve completely destroying the nerve tissue or stopping the function of one or more nerves while allowing one or more other nerves in the same area to maintain their function. Typically, the systems of the present disclosure will contact the mucosa overlying one or more of these one or more nerves, and energy will be delivered through the mucosa to neuromodulate the nerves. In some embodiments, the treatments of the present disclosure may involve disruption of parasympathetic or sensory nerve signals in one or more nerves (e.g., the posterior nasal nerve). In some embodiments, the destruction of submucosal tissue, as discussed above, may also change or disrupt the functions of the nerves in the same region (e.g., the posterior nasal region).

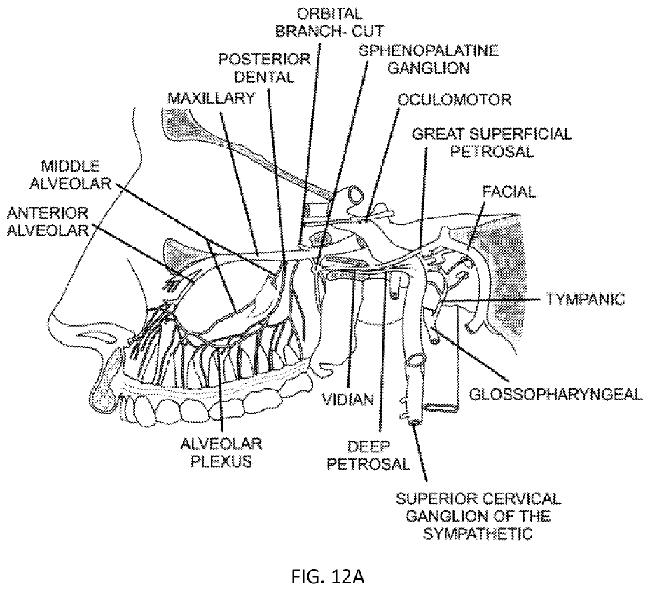

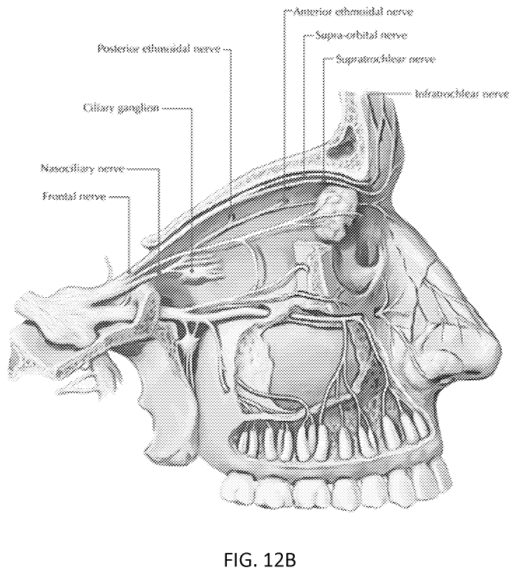

FIGS. 12A and 12B are sagittal views of a human nasal cavity, illustrating the lateral wall and nasal nerves that are related to the treatment of migraines or headaches. Referring to FIG. 12A, a sagittal cross-section of a human nasal cavity is shown. In this cross-sectional view, bone has been removed to illustrate the path of multiple different nerves, such as the vidian nerve and the sphenopalatine ganglion. According to various embodiments and examples described herein, any nerve or combination of nerves may be treated, such as but not limited, to the vidian nerve, vidian nerve branches, the sphenopalatine ganglion or any nerve branching from the sphenopalatine ganglion, such as the posterior nasal nerves (PNN), the posterolateral nasal nerves (PLNN), the posterior inferior lateral nasal nerves, and the posterior superior lateral nasal nerves (descending from the sphenopalatine ganglion in FIG. 12A but not labeled).

In reference to FIG. 12B, in some embodiments, nerves in the nasal cavity that can be treated include, but are not limited to, the frontal nerve, nasociliary nerve, ciliary ganglion, posterior nasal nerve, posterior ethmoidal nerve, supra-orbital nerve, infratrochlear nerve, olfactory nerve, trigeminal nerve, nasopalatine nerve, maxillary nerve, sphenopalatine ganglion, anterior nasal nerves, and the anterior ethmoidal nerve and its branches.

In some embodiments, the present disclosure includes neuromodulation of nerves in the posterior nasal region, such as posterior nasal nerves (PNN), the posterolateral nasal nerves (PLNN), the posterior inferior lateral nasal nerves, and the posterior superior lateral nasal nerves.

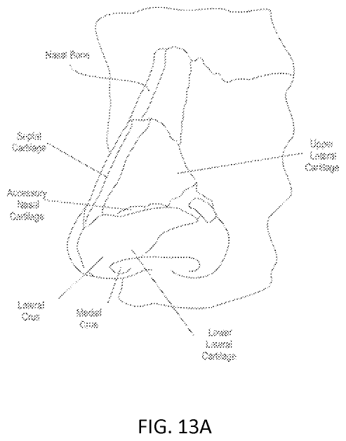

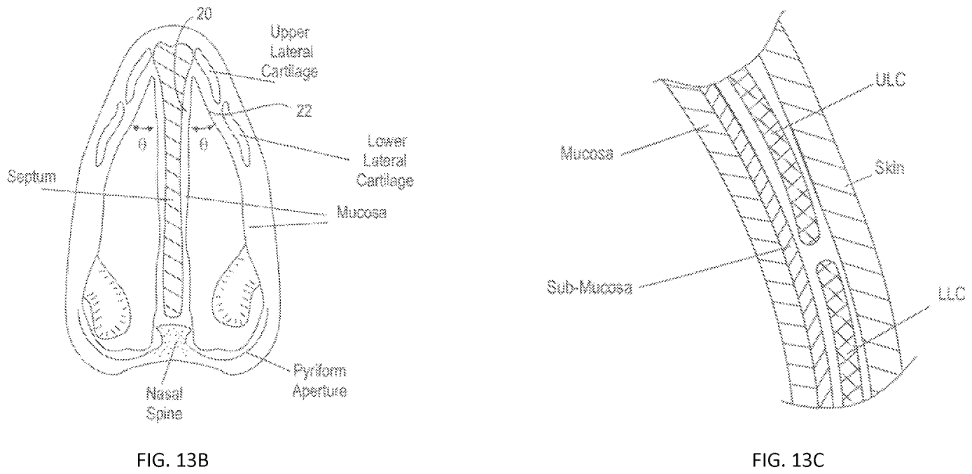

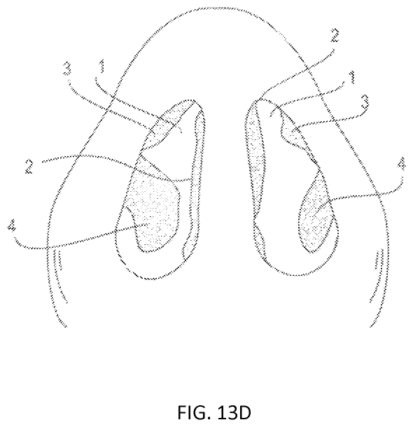

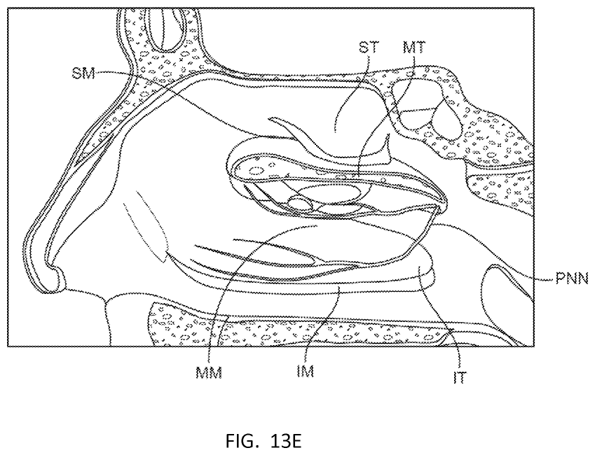

In some embodiments, the systems and methods of the present disclosure can be used to apply energy to reshape one or more tissues in the nasal cavity to treat one or more conditions described herein. FIGS. 13A, 13B, 13C, and 13D illustrate anatomical elements of a human nose. The lower lateral cartilage (LLC) includes an external component referred to as the lateral crus and an internal component referred to as the medial crus. The medial crus and septal nasal cartilage create a nasal septum that separates the left and right nostrils. Upper lateral cartilage lies between the lower lateral cartilages and the nasal bone. The left ULC is separated from the right ULC by the septal cartilage extending down the bridge of the nose. The opposing edges of the LLC and ULC may move relative to one another. Disposed between the opposing edges is an accessory nasal cartilage. The septal nasal cartilage and the ULC form an angle (THETA) called the nasal valve angle.

FIG. 13C illustrates a detailed cross-section of a segment of nose tissue in the area of the intersection of the ULC and the LLC. As shown in FIG. 13C and the detailed view of FIG. 13B, both inner and outer surfaces of the nasal cartilage are covered with soft tissue including mucosa, sub-mucosa and skin. FIG. 13D illustrates a view of the nose as seen from the nostrils. FIG. 13D depicts a nasal valve 1 shown between a septum 2 and an upper lateral cartilage 3. FIG. 13D also depicts the position of a turbinate 4.

In reference to FIG. 13E, three bony structures covered in mucosal tissue, called the superior, middle and inferior nasal turbinates, extend inwardly from each of the two lateral walls of the nasal cavity. FIG. 13E shows the right lateral wall of the nasal cavity, including the superior turbinate ST, superior meatus SM, a cut away middle turbinate MT, middle meatus MM, inferior turbinate IT and inferior meatus IM. The downwardly curved edge of each turbinate defines a passageway below each turbinate, known as a meatus. The inferior meatus is located beneath the inferior turbinate, the middle meatus is located beneath the middle turbinate, and the superior meatus is located beneath the superior turbinate.

In some embodiments, the devices of the present disclosure can be used to conformationally change the structure of one or more of these tissues of the nasal cavity.

For example, the systems of the present disclosure can be used to reshape the internal or external nasal valve anatomy to allow greater airflow without necessarily expanding the diameter of the nasal passage. In some embodiments, energy may be delivered into the nasal valve, submucosal tissue, lateral walls of the nasal cavity, nasal septum, the inferior anterior, and middle turbinates, or cartilage tissue to cause a conformational change or a change in the physical properties of the submucosal tissue or cartilage. For example, in some embodiments, energy may be applied to a targeted region of tissue adjacent to a nasal valve such that the tissue modification results in a tightening, shrinking, or enlarging of such targeted tissues resulting in a change of shape. For example, energy delivery may be accomplished by transferring the energy through the tissue covering the submucosa or cartilage such as the epithelium, mucosa, sub-mucosa, muscle, ligaments, tendon or skin.

In some embodiments, the tissue of the internal or external nasal valve or surrounding tissues may be strengthened or otherwise modified to resist a conformational change in response to the negative pressure of inspiration. In some embodiments, this strengthening may be performed by applying treatments selected to change mechanical or structural properties of the treated tissue. In some embodiments, such treatments may include the application of energy to selected regions of nasal valve or surrounding tissues.

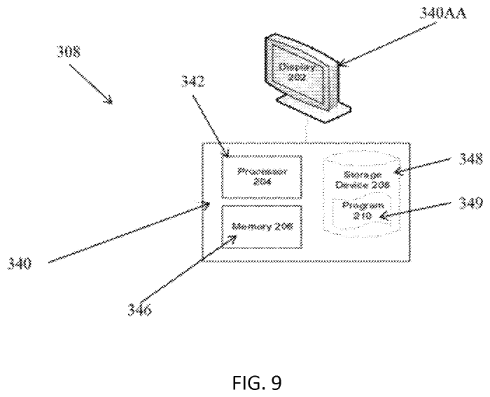

FIG. 9 shows, by way of example, a diagram of a processing architecture 308, which may be used in connection with the methods and systems of the present disclosure. A computer processing device 340 can be coupled to display 340AA for graphical output. Processor 342 can be a computer processor 342 capable of executing software. Typical examples can be computer processors (such as Intel® or AMD® processors), ASICs, microprocessors, and the like. Processor 342 can be coupled to memory 346, which can be typically a volatile RAM memory for storing instructions and data while processor 342 executes. Processor 342 may also be coupled to storage device 348, which can be a non-volatile storage medium, such as a hard drive, FLASH drive, tape drive, DVDROM, or similar device. Although not shown, computer processing device 340 typically includes various forms of input and output. The I/O may include network adapters, USB adapters, Bluetooth radios, mice, keyboards, touchpads, displays, touch screens, LEDs, vibration devices, speakers, microphones, sensors, or any other input or output device for use with computer processing device 340. Processor 342 may also be coupled to other type of computer-readable media, including, but are not limited to, an electronic, optical, magnetic, or other storage or transmission device capable of providing a processor, such as the processor 342, with computer-readable instructions. Various other forms of computer-readable media can transmit or carry instructions to a computer, including a router, private or public network, or other transmission device or channel, both wired and wireless. The instructions may comprise code from any computer-programming language, including, for example, C, C++, C#, Visual Basic, Java, Python, Perl, and JavaScript.

Program 349 can be a computer program or computer readable code containing instructions or data, and can be stored on storage device 348. The instructions may comprise code from any computer-programming language, including, for example, C, C++, C#, Visual Basic, Java, Python, Perl, and JavaScript. In a typical scenario, processor 204 may load some or all of the instructions or data of program 349 into memory 346 for execution. Program 349 can be any computer program or process including, but not limited to web browser, browser application, address registration process, application, or any other computer application or process. Program 349 may include various instructions and subroutines, which, when loaded into memory 346 and executed by processor 342 cause processor 342 to perform various operations, some or all of which may effectuate the methods for managing medical care disclosed herein. Program 349 may be stored on any type of non-transitory computer readable medium, such as, without limitation, hard drive, removable drive, CD, DVD or any other type of computer-readable media.

In some embodiments, the computer system may be programmed to perform the steps of the methods of the present disclosure and control various parts of the instant systems to perform necessary operation to achieve the methods of the present disclosure.

The following examples are put forth so as to provide those of ordinary skill in the art with a complete disclosure and description of how to make and use the assay, screening, and therapeutic methods of the disclosure, and are not intended to limit the scope of what the inventors regard as their disclosure.

Background: Sensory and autonomic nerves supply the sinonasal mucosa, and contribute to the pathophysiology of certain forms of chronic rhinitis, rhinosinusitis, and craniofacial pain. The compositions of these intranasal nerves have been incompletely studied. The purpose of this cadaveric study was to investigate the relative areas of sensory, parasympathetic, and sympathetic nerve fibers within different nasal nerves.

### NON-LIMITING EXAMPLES

Methods: Ten fresh cadaver heads were dissected, and anterior ethmoid (AEN), posterior nasal (PNN), and posterolateral (PLNN) sections were harvested unilaterally via endonasal and transorbital approaches. Specimens were formalin-fixed, sectioned, and stained with hematoxylin and eosin, as well as for neuropeptides (substance-P, calcitonin gene-related peptide, neurokinins-A and B, vasointestinal peptide, neuropeptide Y), and enzymes (choline acetyltransferase and tyrosine hydroxylase). Enzyme and neuropeptide nerve marker percent areas were calculated using brightfield analysis. Sensory and autonomic nerve marker areas were then compared within and between AENs, PNNs, and PLNNs.

Results: In total, 10 PNNs and AENs, and 8 PLNNs were available for analyses. Sensory, parasympathetic, and sympathetic nerve markers were identified in every PNN, PLNN, and AEN, and were mostly equivalent between nerves. Only neurokinin-A demonstrated a significantly greater percent area than other markers across different nasal nerves.

Conclusion: Sensory and autonomic nerve markers were present in all AENs, PNNs, and PLNNs, and were largely equivalent between nerves. NKA presented the greatest percent area consistently across each of the nerve types. Relative contributions of sensory versus of autonomic dysfunction can relate to chronic rhinitis, rhinosinusitis, and craniofacial pain.

### Introduction

Activation of or aberrations in intranasal sensory and autonomic nerves may play significant roles in the pathophysiology of some forms of chronic rhinitis (CR), rhinosinusitis, and craniofacial pain (CFP). While research is ongoing to determine the exact mechanisms behind intranasal nerve function or dysfunction in these conditions, it is critical to understand what nerve types supply different nasal cavity regions and how they contribute to sinonasal pathology. Taken further, appreciating sensory and autonomic fiber compositions of these intranasal nerves would be helpful in furthering the understanding of different sinonasal conditions, outcomes of certain treatment options, and for exploring novel therapeutic interventions. However, nasal nerve sensory and autonomic compositions have been incompletely studied.

Regarding intranasal autonomic innervation, it has been established that the vidian nerve supplies at least the posterior sinonasal cavities with both sympathetic and parasympathetic fibers by way of the posterior nasal nerve (PNN) and posterolateral nasal nerves (PLNNs) that emanate from the pterygopalatine ganglion (PPG) and traverse foraminae in the palatine bone. Autonomic nerve fibers were also recently demonstrated in the anterior ethmoid nerve (AEN) in fresh cadavers as well, and these would presumably supply more anterior portions of the sinonasal cavities.

Regarding sinonasal sensory innervation, there is less data on the exact neural pathways supplying different intranasal regions. Most of the published literature centers on gross anatomic dissection, without histologic confirmation of nerve fiber type(s). For example, anatomic studies have shown that the AEN and posterior ethmoid nerve (PEN) supply the sinonasal cavities by way of the trigeminal nerve's ophthalmic division (V1). Some other anatomic studies imply that the trigeminal nerve's maxillary division (V2) provides innervation to the nasal mucosa via the PNN. However, there has been very little published with regard the sensory fiber composition of the intranasal nerves. The purpose of this study was to analyze the relative sensory and autonomic nerve fiber contributions to cadaveric AENs, PNNs, and PLNNs.

### Methods

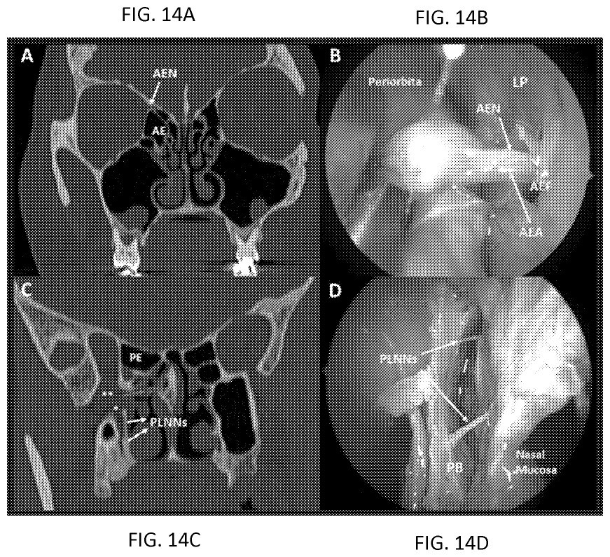

Ten fresh latex-injected cadaver heads were dissected. Cadaver age, gender, and ethnicity were recorded. It was determined that no oversight was required by the Henry Ford Health Institutional Review Board for this study. Transnasal and orbital dissections were performed unilaterally to harvest approximately 1 cm sections of PNNs, PLNNs, and AENs (FIGS. 14A-14D). The PNN was defined as the nerve coursing with the sphenopalatine artery exiting the sphenopalatine foramen. Nerves were considered PLNNs if they coursed through palatine bone foramina separate from the sphenopalatine foramen. The AEN was defined as the nerve coursing with the anterior ethmoid artery entering the anterior ethmoid foramen.

FIGS. 14A-14D shows computed tomography (CT) and endoscopic images to illustrate the nasal nerves that were sampled for this study. FIG. 14A shows a coronal CT depicting the right anterior ethmoid nerve (AEN) coursing through the anterior ethmoid foramen (AEF) (tip of the arrow shown in FIG. 14A) to supply portions of the anterior ethmoid sinus (AE). FIG. 14B shows a right transorbital approach to harvest a segment of the AEN running with the anterior ethmoid artery (AEA), from the periorbita to the anterior ethmoid foramen (AEF). FIG. 14C shows a coronal CT depicting the approximate positions of the right posterior nasal nerve (PNN) emanating from the sphenopalatine foramen (arrow labeled PNN) and right posterolateral nasal nerves (PLNNs) coursing from palatine bone foramina (arrows labeled PLNN) to supply the nasal mucosa. The pterygopalatine fossa (PPF) (double asterisk) and descending palatine nerve (single asterisk) are also highlighted. FIG. 14D shows a right transnasal approach showing the PNN coursing with the sphenopalatine artery (SPA) from the sphenopalatine foramen, and two PLNNs coursing from palatine bone (PB) foramina to the overlying nasal mucosa. LP, lamina papyracea; PE, posterior ethmoid sinus

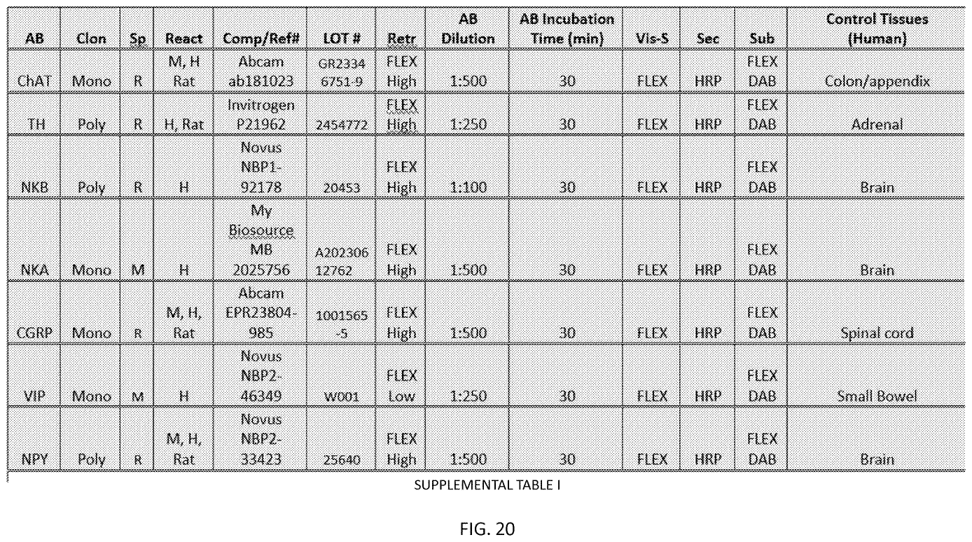

Specimens were formalin-fixed, sectioned at 4 microns, placed on autofrost immunohistochemistry (IHC) slides, and stained with hematoxylin and eosin. Using the Agilent Autostainer platform (Agilent Technologies, Santa Clara, CA, USA), IHC staining was performed for choline acetyltransferase (ChAT), tyrosine hydroxylase (TH), and the following neuropeptides: substance-P (SP), calcitonin gene-related peptide (CGRP), neurokinins-A and B (NKA, NKB), vasointestinal peptide (VIP), and neuropeptide Y (NPY). SP, CGRP, NKA, and NKB represented sensory markers. ChAT and VIP represented parasympathetic markers. TH and NPY represented sympathetic markers. Details of each antibody, its preparation, and control tissues used for validation are presented in Supplemental Table I (see FIG. 20).

Separate positive and negative tissue control slides were stained on the same run as the sample tissue slides for each antibody. Slide preparation for IHC staining started with incubating slides in a 60° C. slide oven for 1 hour. The slides were then rehydrated through xylene, 100% reagent alcohol, 95% reagent alcohol, and DiH2O before performing a heat-induced epitope retrieval in the Agilent PT Link chamber with either Agilent High or Low pH Target Retrieval Solution. Retrieval was performed at 98° C. for 20 minutes. The slides were placed directly in Agilent Wash Buffer (K8007) for 5 minutes before placing in the Autostainer for automated staining. Digital images of processed slides were created and uploaded electronically for subsequent marker analyses.

When nerve tissue was identifiable on light microscopy, nerve marker areas were assessed with brightfield analysis using Python v3.9 (open source). Thresholds for each of the channels was accomplished through color separation, using color vectors for each channel in the red-blue-green images. Color vectors were determined by assessing positive and negative controls and test nerve samples to minimize background and maximize detection of positive marker staining. After thresholding, the generated masks were used to measure the area occupied by positive nerve marker signals, which were then divided by the total tissue area to calculate nerve marker percent areas for each marker in each nasal nerve type.

Statistical analysis was performed using Stata v17 (College Station, TX, USA). Nerve marker areas were described as means and standard deviations for AENs, PNNs, and PLNNs. To determine whether any outliers existed within the data, the Grubbs test for outliers (extreme Studentized deviant) was performed on percent marker area for each nasal nerve and marker type separately (11 were found). To ensure outlier identification, the Rosner test was also run, which identified the same 11 outliers, plus 6 more (17 total). After excluding all 17 outliers, exploratory analyses were performed. First, a two-way ANOVA was performed to assess whether nerve type (AEN, PNN, PLNN), nerve marker (enzymes and neuropeptides), and nerve-marker interaction predicted nerve marker percent areas in specimens. Next, a series of follow-up one-way ANOVAs was performed to explore the results of the two-way ANOVA more deeply. Each follow-up one-way ANOVA implemented Bonferroni correction to control the family-wise error rate and minimize the risk of false-positives. First, a one-way ANOVA was performed for each nerve marker across all nasal nerves to determine whether individual nerve marker percent area varied across all nerve types (e.g., TH percent area compared across all AENs, PNNs, and PLNNs). Subsequently, for each marker, one-way ANOVA pairwise comparisons were made between individual nasal nerves (e.g., TH percent area compared separately between AEN and PNN, AEN and PLNN, and PNN and PLNN). Lastly, for each nasal nerve type, one-way ANOVA pairwise comparisons were made between each nerve marker (e.g., for AENs, PNNs, and PLNNs, each marker's percent area was compared to every other marker's percent area).

### Results

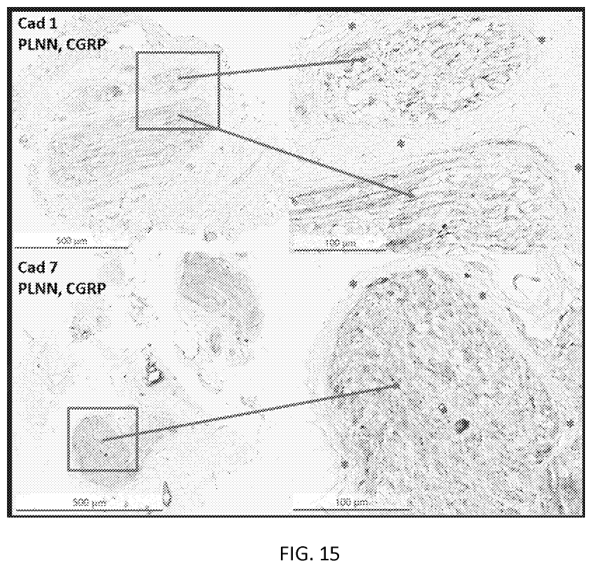

Of the 10 cadavers, mean age was 73.0±10.6 years (range=57-85), 30% were female, and 90% were White (one Pacific islander). Underlying medical conditions were not available. Note that due to concerns with suboptimal stain integrity, VIP-stained specimens were excluded from analyses. Of 172 available marker-stained nerve samples, 139 were used for statistical analyses (after excluding outliers and VIP-stained specimens). FIGS. 15-19D show representative nerve marker staining of different nasal nerves that were used for comparative analyses.

Table I shows mean values of individual nerve marker areas, total tissue areas, and marker percent areas across AENs, PNNs, and PLNNs. Each marker represented between about 1-13% of total tissue area in each nerve specimen, with NKA demonstrating the greatest percent areas (5.2-13.2%), followed by SP (0.9-6.3%). Other markers generally represented about 1-3% of total tissue areas. While there were relatively wide variations in marker and tissue areas, there was lower variability in marker percent area, showing that marker and tissue areas generally varied proportionately.

Table I includes mean nerve marker area, total nerve tissue area, and percent marker area in each nasal nerve type (AEN, anterior ethmoid nerve; PNN, posterior nasal nerve; PLNN, posterolateral nasal nerves) (TH, tyrosine hydroxylase; ChAT, choline acetyltransferase; NPY, neuropeptide Y; CGRP, calcitonin gene-related peptide; NKA, neurokinin A; NKB, neurokinin B; SP, substance-P.

TABLE I

| | | | | | | | Percent | |
|---|---|---|---|---|---|---|---|---|
| | | | Marker Area | | Total Tissue Area | | Marker Area | |
| Nerve | Nerve | Sample | (μm²) | | (μm²) | | (%) | |
| Type | Marker | Size | Mean | SD | Mean | SD | Mean | SD |
| AEN | TH | 7 | 9,140 | 7,624 | 518,540 | 289,495 | 1.50 | 0.94 |
| | ChAT | 8 | 12,634 | 12,754 | 6,009,560 | 13,484,122 | 0.89 | 0.82 |
| | NPY | 4 | 5,966 | 6,117 | 12,705,649 | 24,219,777 | 0.43 | 0.33 |
| | CGRP | 3 | 11,685 | 9,947 | 451,304 | 176,031 | 2.24 | 1.42 |
| | NKA | 7 | 123,984 | 147,789 | 904,600 | 535,010 | 12.81 | 9.97 |
| | NKB | 4 | 4,749 | 1,862 | 1,060,678 | 1,077,806 | 0.85 | 0.53 |
| | SP | 10 | 72,615 | 83,777 | 1,144,541 | 851,111 | 6.25 | 5.28 |
| PNN | TH | 9 | 3,5172 | 31,802 | 1,962,968 | 1,572,313 | 2.51 | 2.34 |
| | ChAT | 9 | 48,477 | 22,085 | 2,566,936 | 1,644,430 | 2.54 | 1.56 |
| | NPY | 9 | 20,801 | 16,512 | 2,386,301 | 2,284,406 | 1.53 | 1.71 |
| | CGRP | 8 | 16,457 | 11,589 | 2,532,282 | 4,099,636 | 1.29 | 0.72 |
| | NKA | 9 | 129,372 | 129,372 | 2,736,386 | 1,394,615 | 5.23 | 2.32 |
| | NKB | 7 | 16,504 | 12,267 | 1,892,918 | 1,141,334 | 0.87 | 0.64 |
| | SP | 7 | 41,728 | 32,446 | 3,489,451 | 1,786,589 | 1.16 | 0.66 |
| PLNN | TH | 6 | 10,762 | 8,028 | 470,478 | 241,988 | 2.65 | 2.10 |
| | ChAT | 8 | 11,531 | 14,062 | 482,231 | 530,208 | 2.64 | 2.27 |
| | NPY | 4 | 3,421 | 1,825 | 348,729 | 122,832 | 0.94 | 0.42 |
| | CGRP | 6 | 7,692 | 3,095 | 394,718 | 289,240 | 2.32 | 1.20 |
| | NKA | 5 | 56,570 | 78,697 | 379,107 | 351,633 | 13.18 | 6.26 |
| | NKB | 4 | 11,095 | 6,934 | 1,324,332 | 1,661,782 | 1.67 | 0.99 |
| | SP | 5 | 5,085 | 4,542 | 576,451 | 466,622 | 0.90 | 0.31 |

As an example, FIG. 15 shows representative intraneural staining with CGRP in PLNNs across different cadavers.

FIG. 15 shows examples of immunostaining for calcitonin gene-related peptide (CGRP) in posterolateral nasal nerve (PLNN) specimens in two different cadavers. Magnified views of the nerve specimens are shown to the right of their respective lower magnification views. The brown staining shows that the CGRP was localized intraneurally, deep to surrounding perineurium (asterisks).

Supplemental Table II shows the results of the two-way ANOVA assessing whether nerve type, nerve marker, and nerve type-to-marker interaction predicted the marker percent areas seen in nerve specimens. The model showed that nerve markers and the interaction between nerve type and marker predicted marker percent areas in specimens (p<0.0004). Of note, nerve type by itself was not significantly associated with predicting marker percent areas (p=0.062).

Supplemental Table III shows the results of the one-way ANOVA assessing whether marker areas varied across any of the three nerve types. While SP demonstrated some potential variation across all nerves (p=0.013), and NKA also had a p-value approaching uncorrected significance (p=0.052), these variations were not significant after Bonferroni correction. No other nerve markers varied significantly across all the nasal nerves.

When nerve marker areas were compared in pairwise groupings between AENs, PNNs, and PLNNs, some limited variation may have been present. For example, NKA and SP showed some trend towards difference between two of the three nerve pairwise comparisons (p<0.05), but these p-values did not remain significant after Bonferroni correction.

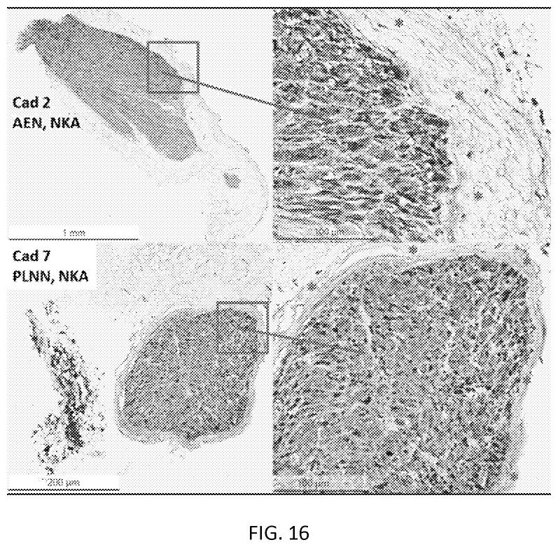

FIG. 16 shows representative intraneural NKA staining being equivalent across different nasal nerve types in different cadavers. FIG. 16 shows examples of immunostaining for neurokinin-A (NKA) in different nasal nerve types across two different cadavers. Magnified views of the nerve specimens are shown to the right of their respective lower magnification views. The brown staining shows that the NKA was localized intraneurally, deep to surrounding perineurium (asterisks). AEN, anterior ethmoid nerve; PLNN, posterolateral nasal nerve.

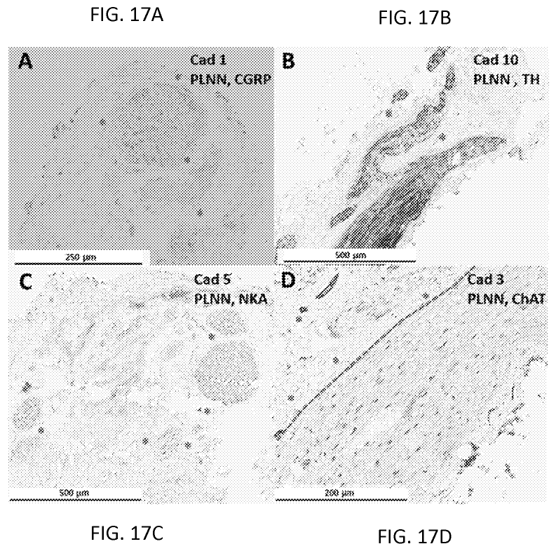

FIGS. 17A-19D show representative staining of different markers across one or more nasal nerves in different cadavers.

FIGS. 17A-17D show examples of immunostaining for different neuropeptides in posterolateral nasal nerves (PLNN) across different cadavers. Note the brown staining intraneurally representing the given nerve marker, deep to surrounding perineurium (red asterisks). FIG. 17A shows calcitonin gene-related peptide (CGRP). FIG. 17B shows tyrosine hydroxylase (TH). FIG. 17C shows neurokinin-A (NKA). FIG. 17D shows choline acetyltransferase (ChAT), nerve tissue is seen to the right of the dotted line, while perineurium and other connective tissue (red asterisks) are seen to the left of the line.

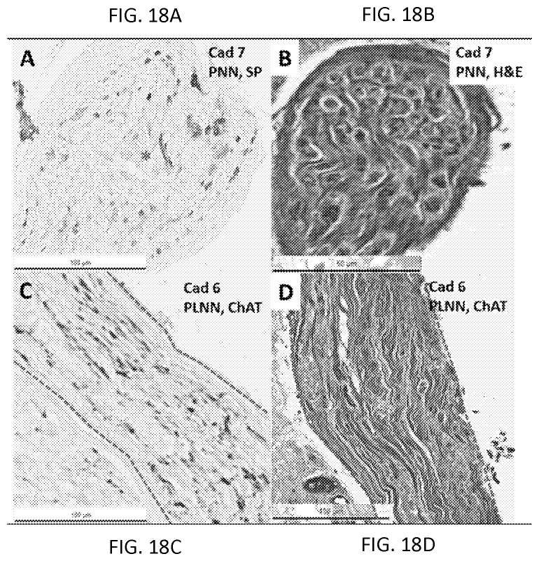

FIGS. 18A-18D show a comparison of neuropeptide staining relative to the appearance of the same nerves on hematoxylin and eosin staining. FIG. 18A shows substance-P (SP) in cadaver 7's posterior nasal nerve (PNN), with asterisks representing nerve tissue. FIG. 18B shows cadaver 7's hematoxylin and eosin stain highlighting the nerve tissue (red asterisk) corresponding to the region of SP staining in (FIG. 18A). FIG. 18C shows choline acetyltransferase (ChAT) in cadaver 6's posterolateral nasal nerve (PLNN), with nerve tissue being outline with dotted lines. FIG. 18D shows cadaver 6's hematoxylin and eosin stain highlighting the nerve tissue (outlined by dotted lines) that corresponds to the region of ChAT staining in (FIG. 18C).

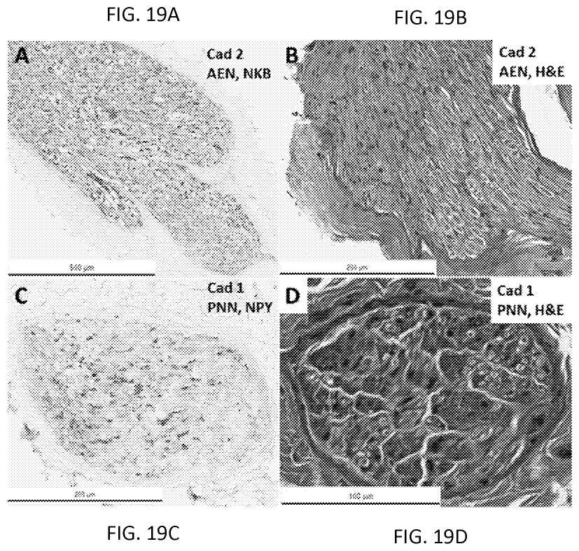

FIGS. 19A-19D show a comparison of neuropeptide staining relative to the appearance of the same nerves on hematoxylin and eosin staining. FIG. 19A shows neurokinin-B (NKB) in cadaver 2's anterior ethmoid nerve (AEN), with asterisks representing nerve tissue. FIG. 19B shows cadaver 2's hematoxylin and eosin stain highlighting the nerve tissue (red asterisk) corresponding to the region of NKB staining in (FIG. 19A). FIG. 19C shows neuropeptide-Y (NPY) in cadaver 1's posterior nasal nerve (PNN). FIG. 19D shows cadaver 1's hematoxylin and eosin stain highlighting the nerve tissue (red asterisk) that corresponds to the region of NPY staining in (FIG. 19C).

Table II shows pairwise comparisons of nerve marker areas between each marker, within each nasal nerve. NKA was the only marker with percent areas that were significantly greater than some of the other markers even after Bonferroni correction (e.g., NKA>CGRP, NKB, and SP in PNNs). NKA also demonstrated a consistently greater percent area compared to multiple markers across each nerve type (p<0.05 in 19 of 21 comparisons). However, the NKA associations in AENs and PLNNs were not significant after Bonferroni correction.

Table II shows pairwise comparisons of nerve marker areas within individual nasal nerves. Bold non-italicized p-values were <0.05, but were not significant after Bonferroni correction (p≤0.0008). Bold italicized underlined values were significant after Bonferroni correction (AEN, anterior ethmoid nerve; PNN, posterior nasal nerve; PLNN, posterolateral nasal nerves; TH, tyrosine hydroxylase; ChAT, choline acetyltransferase; NPY, neuropeptide Y; CGRP, calcitonin gene-related peptide; NKA, neurokinin A; NKB, neurokinin B; SP, substance-P. SS, sum of squares; DF, degrees of freedom; MS, mean squares; F, F-statistic).

TABLE II

| | Marker | Marker | | Unadjusted | | | | | |
|---|---|---|---|---|---|---|---|---|---|
| Nerve | 1 | 2 | N | R-squared | Partial SS | DF | MS | F | P-value* |
| AEN | TH | ChAT | 15 | 0.118 | 1.357 | 1 | 1.357 | 1.77 | 0.207 |
| | | NPY | 11 | 0.337 | 2.881 | 1 | 2.882 | 4.58 | 0.061 |
| | | CGRP | 10 | 0.111 | 1.167 | 1 | 1.167 | 1.00 | 0.347 |
| | | NKA | 14 | 0.427 | 448.256 | 1 | 448.256 | 8.95 | 0.011 |
| | | NKB | 11 | 0.149 | 1.076 | 1 | 1.076 | 1.57 | 0.242 |
| | | SP | 17 | 0.266 | 93.073 | 1 | 93.073 | 5.44 | 0.034 |
| | ChAT | NPY | 12 | 0.102 | 0.567 | 1 | 0.567 | 1.14 | 0.312 |
| | | CGRP | 11 | 0.313 | 3.967 | 1 | 3.967 | 4.11 | 0.073 |
| | | NKA | 15 | 0.469 | 530.443 | 1 | 530.443 | 11.48 | 0.005 |
| | | NKB | 12 | 0.001 | 0.006 | 1 | 0.006 | 0.01 | 0.919 |
| | | SP | 18 | 0.333 | 127.555 | 1 | 127.555 | 7.97 | 0.012 |
| | NPY | CGRP | 7 | 0.563 | 5.613 | 1 | 5.613 | 6.43 | 0.052 |
| | | NKA | 11 | 0.396 | 390.186 | 1 | 390.186 | 5.89 | 0.038 |
| | | NKB | 8 | 0.226 | 0.343 | 1 | 0.343 | 1.75 | 0.234 |
| | | SP | 14 | 0.278 | 96.722 | 1 | 96.722 | 4.61 | 0.053 |
| | CGRP | NKA | 10 | 0.281 | 234.686 | 1 | 234.686 | 3.13 | 0.115 |
| | | NKB | 7 | 0.407 | 3.339 | 1 | 3.339 | 3.43 | 0.123 |
| | | SP | 13 | 0.127 | 37.086 | 1 | 37.086 | 1.60 | 0.232 |
| | NKA | NKB | 11 | 0.379 | 364.529 | 1 | 364.530 | 5.50 | 0.044 |
| | | SP | 17 | 0.173 | 177.339 | 1 | 177.339 | 3.14 | 0.097 |
| | NKB | SP | 14 | 0.249 | 83.448 | 1 | 83.448 | 3.97 | 0.070 |
| PNN | TH | ChAT | 18 | 0.0001 | 0.005 | 1 | 0.005 | 0.00 | 0.973 |
| | | NPY | 18 | 0.061 | 4.332 | 1 | 4.332 | 1.03 | 0.325 |
| | | CGRP | 17 | 0.118 | 6.352 | 1 | 6.352 | 2.01 | 0.176 |
| | | NKA | 18 | 0.277 | 33.230 | 1 | 33.230 | 6.13 | 0.025 |
| | | NKB | 16 | 0.187 | 10.600 | 1 | 10.600 | 3.22 | 0.094 |
| | | SP | 16 | 0.134 | 7.151 | 1 | 7.151 | 2.17 | 0.163 |
| | ChAT | NPY | 18 | 0.097 | 4.618 | 1 | 4.618 | 1.72 | 0.208 |
| | | CGRP | 17 | 0.225 | 6.686 | 1 | 6.686 | 4.35 | 0.055 |
| | | NKA | 18 | 0.342 | 32.456 | 1 | 32.456 | 8.32 | 0.011 |
| | | NKB | 16 | 0.336 | 11.016 | 1 | 11.016 | 7.07 | 0.019 |
| | | SP | 16 | 0.254 | 7.492 | 1 | 7.492 | 4.77 | 0.047 |
| | NPY | CGRP | 17 | 0.009 | 0.251 | 1 | 0.251 | 0.14 | 0.715 |
| | | NKA | 18 | 0.481 | 61.559 | 1 | 61.559 | 14.80 | 0.001 |
| | | NKB | 16 | 0.062 | 1.713 | 1 | 1.713 | 0.93 | 0.352 |
| | | SP | 16 | 0.020 | 0.529 | 1 | 0.529 | 0.28 | 0.603 |
| | CGRP | NKA | 17 | 0.585 | 65.816 | 1 | 65.816 | 21.12 | 0.0003 |
| | | NKB | 15 | 0.096 | 0.647 | 1 | 0.647 | 1.38 | 0.262 |
| | | SP | 15 | 0.009 | 0.057 | 1 | 0.057 | 0.12 | 0.738 |
| | NKA | NKB | 16 | 0.622 | 74.788 | 1 | 74.788 | 23.01 | 0.0003 |
| | | SP | 16 | 0.588 | 65.065 | 1 | 65.065 | 19.94 | 0.0005 |
| | NKB | SP | 14 | 0.056 | 0.301 | 1 | 0.301 | 0.72 | 0.414 |
| PLNN | TH | ChAT | 14 | 0.0000 | 0.001 | 1 | 0.001 | 0.00 | 0.990 |
| | | NPY | 10 | 0.238 | 7.040 | 1 | 7.040 | 2.50 | 0.152 |
| | | CGRP | 12 | 0.011 | 0.327 | 1 | 0.327 | 0.11 | 0.745 |
| | | NKA | 11 | 0.629 | 302.484 | 1 | 302.484 | 15.25 | 0.004 |
| | | NKB | 10 | 0.085 | 2.320 | 1 | 2.320 | 0.74 | 0.414 |
| | | SP | 11 | 0.271 | 8.326 | 1 | 8.326 | 3.35 | 0.101 |
| | ChAT | NPY | 12 | 0.173 | 7.681 | 1 | 7.681 | 2.10 | 0.178 |
| | | CGRP | 14 | 0.008 | 0.340 | 1 | 0.340 | 0.09 | 0.764 |
| | | NKA | 13 | 0.640 | 342.273 | 1 | 342.273 | 19.55 | 0.001 |
| | | NKB | 12 | 0.060 | 2.497 | 1 | 2.497 | 0.64 | 0.443 |
| | | SP | 13 | 0.202 | 9.227 | 1 | 9.227 | 2.78 | 0.124 |
| | NPY | CGRP | 10 | 0.372 | 4.587 | 1 | 4.587 | 4.74 | 0.061 |
| | | NKA | 9 | 0.680 | 333.15472 | 1 | 333.155 | 14.85 | 0.006 |
| | | NKB | 8 | 0.234 | 1.0644939 | 1 | 1.065 | 1.83 | 0.225 |
| | | SP | 9 | 0.003 | .00264211 | 1 | 0.003 | 0.02 | 0.891 |
| | CGRP | NKA | 11 | 0.663 | 321.75081 | 1 | 321.75081 | 17.69 | 0.002 |
| | | NKB | 10 | 0.091 | 1.0232907 | 1 | 1.0232907 | 0.80 | 0.396 |
| | | SP | 11 | 0.419 | 5.4760972 | 1 | 5.4760972 | 6.48 | 0.031 |
| | NKA | NKB | 9 | 0.649 | 294.63632 | 1 | 294.63632 | 12.93 | 0.009 |
| | | SP | 10 | 0.706 | 376.91299 | 1 | 376.91299 | 19.22 | 0.002 |
| | NKB | SP | 9 | 0.279 | 1.2972167 | 1 | 1.2972167 | 2.71 | 0.144 |

Supplemental Table I (see FIG. 20) shows antibody information, preparation, and control tissues used for validation. Supplemental Table I includes antibody (AB) information, processing protocol, and control tissues used for validation. FLEX High TRS (Target Retrieval Solution) and FLEX Low TRS were used for antigen retrieval (Retr) in the PT Link. Slides were placed in 65° C. solution, heated up to 98° C. and incubated for 20 minutes, then brought back down to 68° C. before removing from the TRS and placed in Wash Buffer for 5 minutes. The FLEX High TRS is a part of Agilent's EnVision FLEX+ HRP Kit (K8002), which includes Hydrogen Peroxide, HRP (Horseradish Peroxidase), DAB (3,3′ Diaminobenzidine), Mouse Linker (when needed), and Wash Buffer. The FLEX Low TRS catalog number is K8005. R, rabbit; M, mouse; H, human; Clon, clonality; Sp, species; React, reactivity; Comp/Ref #, Company/Reference #; Retr, retrieval; Vis-S, Vis System; Sec, secondary; Sub, substrate.

Supplemental Table II shows two-way ANOVA assessing whether nerve types, nerve markers, and nerve-marker interactions predicted the marker percent areas seen in nerve specimens. Bold p-values were statistically significant (AEN, anterior ethmoid nerve; PNN, posterior nasal nerve; PLNN, posterolateral nasal nerves; MSE, mean squared error; SS, sum of squares; DF, degrees of freedom; MS, mean squares; F, F-statistic).

SUPPLEMENTAL TABLE II

| | N = 139 | | R-squared = 0.564 | | |
|---|---|---|---|---|---|
| | Root MSE = 3.224 | | Adjusted R-squared = 0.491 | | |
| Component of Model | Partial SS | DF | MS | F | p-value |
| Overall model | 1588.580 | 20 | 79.429 | 7.64 | <0.0001 |
| Nerve (AEN, PNN, PLNN) | 59.287 | 2 | 29.644 | 2.85 | 0.0617 |
| Nerve marker (enzymes and | 1244.725 | 6 | 207.454 | 19.96 | <0.0001 |
| neuropeptides) | | | | | |
| Nerve-to-nerve marker interaction | 406.750 | 12 | 33.896 | 3.26 | 0.0004 |
| Model residual | 1226.343 | 118 | 10.393 | N/A | N/A |

Supplemental Table III shows one-way ANOVAs comparing nerve marker percent areas versus nerve types for each nerve marker. The bold p-value for substance-P (SP) was <0.05, but was not significant after Bonferroni correction (p≤0.0071) (TH, tyrosine hydroxylase; ChAT, choline acetyltransferase; NPY, neuropeptide Y; CGRP, calcitonin gene-related peptide; NKA, neurokinin A; NKB, neurokinin B. MSE, mean squared error; SS, sum of squares; DF, degrees of freedom; MS, mean squares; F, F-statistic).

Supplemental Table III

| | | Unadjusted | Partial | | | | |
|---|---|---|---|---|---|---|---|
| Markers | N | R-squared | SS | DF | MS | F | p-value* |
| TH | 22 | 0.072 | 5.536 | 2 | 2.768 | 0.74 | 0.490 |
| ChAT | 25 | 0.206 | 15.619 | 2 | 7.809 | 2.86 | 0.079 |
| NPY | 17 | 0.127 | 3.526 | 2 | 1.763 | 1.01 | 0.388 |
| CGRP | 17 | 0.225 | 4.324 | 2 | 2.162 | 2.03 | 0.168 |
| NKA | 21 | 0.279 | 308.460 | 2 | 154.230 | 3.49 | 0.052 |
| NKB | 15 | 0.235 | 1.911 | 2 | 0.955 | 1.84 | 0.201 |
| SP | 22 | 0.367 | 147.462 | 2 | 73.731 | 5.51 | 0.013 |

While embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments described herein can be employed. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification, embodiments have been described in a way which enables a clear and concise specification to be written, but it is intended and will be appreciated that embodiments may be variously combined or separated without parting from the scope of the present disclosure. It is intended that the present disclosure be limited only to the extent required by the appended claims and the applicable rules of law.

As utilized herein, the terms “comprise” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art. The use of the terminology X “or” Y herein should be interpreted as meaning either “X” or “Y” individually, or both “X and Y” together.

All patents and publications mentioned in this specification are herein incorporated by reference to the same extent as if each independent patent and publication was specifically and individually indicated to be incorporated by reference.

It is also to be understood that the following claims are to cover all generic and specific features of the disclosure described herein, and all statements of the scope of the disclosure which, as a matter of language, might be said to fall therebetween.

We claim:

1. A method for treating tissue, the method comprising:
assessing one or more biomarker profiles in nasal tissue, the one or more biomarker profiles being indicative of rhinitis;
advancing a treatment device into a nasal cavity of a patient, the treatment device comprising one or more treatment elements, the one or more treatment elements having an array of energy delivery elements disposed thereon; and
providing treatment, based on the assessed biomarker profiles, to one or more target tissue areas in the nasal cavity by activating one or more of the energy delivery elements of the array of energy delivery elements;
wherein the one or more biomarker profiles relate to tyrosine hydroxylase (TH), choline acetyltransferase (ChAT), neuropeptide Y (NPY), calcitonin gene-related peptide (CGRP), neurokinin A (NKA), neurokinin B (NKB), or substance-P (SP).

2. The method of claim 1, further comprising monitoring the one or more biomarker profiles during the treatment to determine a need for additional treatment with the treatment device.

3. The method of claim 1, further comprising monitoring the one or more biomarker profiles after termination of the treatment to determine a need for additional treatment with the treatment device.

4. The method of claim 1, further comprising monitoring the one or more biomarker profiles after termination of the treatment to access results of the treatment with the treatment device.

5. The method of claim 1, wherein one or more energy delivery elements of the array of energy delivery elements are activated with pulsed electric field energy for conducing pulsed radiofrequency energy or pulsed field ablation.

6. The method of claim 1, wherein one or more energy delivery elements of the array of energy delivery elements are activated with high-voltage direct current pulses of short duration to induce regulated cell death through irreversible electroporation.

7. The method of claim 1, wherein the array of energy delivery elements is positioned in two or more rows on the one or more treatment elements.

8. The method of claim 7, wherein each of the two or more rows comprise at least one pair of energy delivery elements.

9. A method for treating tissue, the method comprising:
assessing one or more biomarker profiles in nasal tissue, the one or more biomarker profiles being indicative of rhinitis;
providing an initial treatment, based on the assessed biomarker profiles, to one or more target tissue areas in a nasal cavity by activating one or more energy delivery elements positioned on a treatment element of a treatment device;
monitoring the one or more biomarker profiles during the initial treatment to track the initial treatment provided with the one or more energy delivery elements of the treatment device; and
providing additional treatment, based on the monitored biomarker profiles during the initial treatment, to the one or more target tissue areas in a nasal cavity,
wherein the one or more biomarker profiles relate to tyrosine hydroxylase (TH), choline acetyltransferase (ChAT), neuropeptide Y (NPY), calcitonin gene-related peptide (CGRP), neurokinin A (NKA), neurokinin B (NKB), or substance-P (SP).

10. The method of claim 9, wherein the one or more energy delivery elements are activated with pulsed electric field energy for conducing pulsed radiofrequency energy or pulsed field ablation.

11. The method of claim 9, wherein the one or more energy delivery elements are activated with high-voltage direct current pulses of short duration to induce regulated cell death through irreversible electroporation.

12. The method of claim 9, wherein the one or more energy delivery elements are positioned in two or more rows on the treatment element.

13. The method of claim 12, wherein each of the two or more rows comprises at least one pair of energy delivery elements.

14. The method of claim 9, further comprising accessing the one or more biomarker profiles after providing the additional treatment.

15. A method for treating tissue, the method comprising:
assessing a first set of biomarker profiles in nasal tissue, the first set of biomarker profiles being indicative of rhinitis;
providing treatment by applying energy to one or more target tissue areas in a nasal cavity with one or more pairs of electrodes;
assessing a second set of biomarker profiles in nasal tissue after providing the treatment to the one or more target tissue areas; and
comparing the first set of biomarker profiles to the second set of biomarker profiles to monitor the treatment to determine success of the treatment in treating rhinitis,
wherein the biomarker profiles relate to tyrosine hydroxylase (TH), choline acetyltransferase (ChAT), neuropeptide Y (NPY), calcitonin gene-related peptide (CGRP), neurokinin A (NKA), neurokinin B (NKB), or substance-P (SP).

16. The method of claim 15, wherein the one or more pairs of electrodes are activated with pulsed electric field energy for conducing pulsed radiofrequency energy or pulsed field ablation.

17. The method of claim 15, wherein the one or more pairs of electrodes are activated with high-voltage direct current pulses of short duration to induce regulated cell death through irreversible electroporation.

18. The method of claim 15, wherein the second set of biomarker profiles being higher than the first set of biomarker profiles indicates success of the treatment.

19. The method of claim 15, further comprising providing additional treatment to the one or more target tissue areas in a nasal cavity when the comparison of the first set of biomarker profiles to the second set of biomarker profiles determines the treatment is unsuccessful.

20. The method of claim 15, further comprising determining success of the treatment when the comparison of the first set of biomarker profiles to the second set of biomarker profiles indicates that the biomarker profiles have increased to above a threshold value or decreased to below a threshold.
